(19) United States
(12) Patent Application Publication
(10) Pub. No.: US 2025/0050087 A1
(43) Pub. Date: Feb. 13, 2025

(54) HEMOSTASIS VALVE

(71) Applicant: Merit Medical Systems, Inc., South Jordan, UT (US)

(72) Inventors: Michael Dean Haslam, Sandy, UT (US); Dillon Despain, Bluffdale, UT (US); Aaron Hopkinson, Herriman, UT (US)

(21) Appl. No.: 18/796,975

(22) Filed: Aug. 7, 2024

Related U.S. Application Data
(60) Provisional application No. 63/518,247, filed on Aug. 8, 2023.

(51) Int. Cl. A61M 39/06; A61M 39/02

(52) U.S. Cl. CPC A61M 39/0613; A61M 2039/027; A61M 2039/064

(57) ABSTRACT

Hemostasis valve devices and methods of use are disclosed. The hemostasis valve devices include a body member, a side-arm, a valve member, and a valve cap. The body member includes a body bore extending therethrough. The body bore includes a proximal bore portion in fluid communication a side-arm bore. In one embodiment, a fluid flow insert is disposed within the body bore in fluid communication with the side-arm bore and the proximal bore portion to direct flushing or priming fluid injected through the fluid flow insert into the proximal bore portion in a circular, spiral, or swirling flow path to remove entrapped air bubbles from the proximal bore portion.

### RELATED APPLICATIONS

This application claims priority to U.S. Provisional Application No. 63/518,247, filed on Aug. 8, 2023 and titled, “Hemostasis Valve Device,” which is hereby incorporated by reference in its entirety.

### TECHNICAL FIELD

The present disclosure relates generally to medical devices. More specifically, the present disclosure relates to medical devices used to access a patient's vascular system. In some embodiments, the present disclosure relates to medical devices used to control blood leakage while accessing the patient's vascular system.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a hemostasis valve device.

FIG. 2 is a side cross-sectional side view of the hemostasis valve device of FIG. 1.

FIG. 3A is a side view of an embodiment of a fluid flow insert of the hemostasis valve device of FIG. 1.

FIG. 3B is a perspective view from a proximal end of the fluid flow insert of FIG. 3A.

FIG. 3C is a another side view of the fluid flow insert of FIG. 3A

FIG. 4 is a perspective view of an embodiment of a fluid flow path of the hemostasis valve device of FIG. 1.

FIG. 5 is a perspective view from a proximal end of another embodiment of a hemostasis valve device.

FIG. 6 is an end view of the hemostasis valve device of FIG. 5.

FIG. 7 is a transverse cross-sectional view from a distal end of the hemostasis valve device of FIG. 5.

FIG. 8 is a side cross-sectional view of the hemostasis valve device of FIG. 5.

FIG. 9 is a perspective view of an embodiment of a fluid flow path of the hemostasis valve device of FIG. 5.

### DETAILED DESCRIPTION

In certain instances, hemostasis valve devices are used to prevent blood leakage and to maintain a position of a medical appliance while accessing a patient's vascular system to treat a patient. For example, treatment of the patient's brain, heart, kidneys, liver, and lungs may be accomplished from an intravascular approach. A catheter may be inserted into the vascular system to provide access to an area of treatment. A hemostasis valve device can be coupled to a proximal end of the catheter. The hemostasis valve device can prevent leakage of blood from the catheter and hold medical appliances, such as a guidewire, that are inserted through the hemostasis valve device into the catheter in a desired position. In some instances, air bubbles entrapped within the hemostasis valve when the hemostasis valve is primed with flush fluid may be displaced into the catheter and into the patient resulting in a severe morbidity or mortality event.

Embodiments herein describe hemostasis valve devices and methods of use thereof. The hemostasis valve devices can be coupled to a catheter inserted into a patient's vascular system for treatment of the patient's brain or other treatment site. In some embodiments within the scope of this disclosure, the hemostasis valve devices include a body member having a side-arm, and a valve member disposed between a valve cap and the body member. A medical appliance, such as a guidewire, can be passed through the valve member into the catheter to prevent blood leakage from the catheter and to hold the medical appliance in a desired position.

In certain embodiments within the scope of this disclosure, a fluid flow insert is disposed within a body member bore distal of the valve member. The fluid flow insert can comprise an external spiral shape fluid flow channel in fluid communication with a side-arm bore and a proximal bore portion of the body member bore disposed between the fluid flow insert and the valve member. The fluid flow channel can be configured to spirally direct flushing fluid from the side-arm bore into the bore proximal portion to flush, dislodge, or remove entrapped air bubbles from the bore proximal portion and/or a distal surface of the valve member. The fluid flow insert can include an insert bore in fluid communication with the proximal bore portion and a distal bore portion.

In another embodiment, the side-arm may be offset relative to a longitudinal axis of the body member such that the side-arm bore communicates with the proximal bore portion offset from the longitudinal axis. This configuration facilitates a spiraling, swirling, or cyclone flow within the proximal bore portion to flush entrapped air bubbles from the proximal bore portion and/or the distal surface of the valve member.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

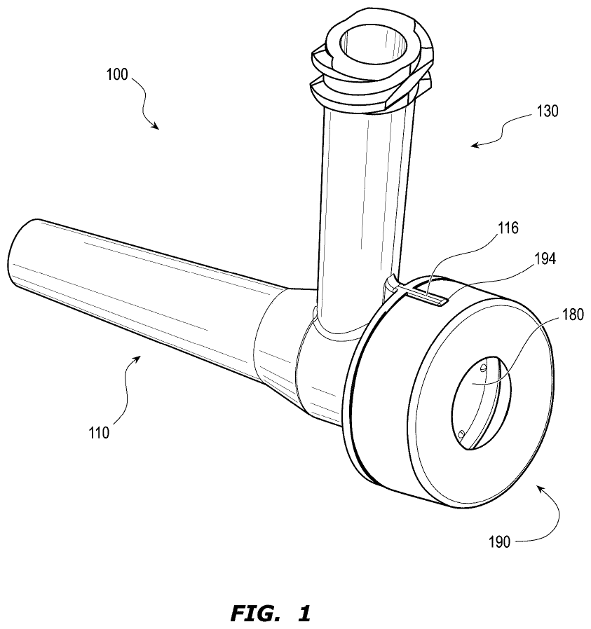

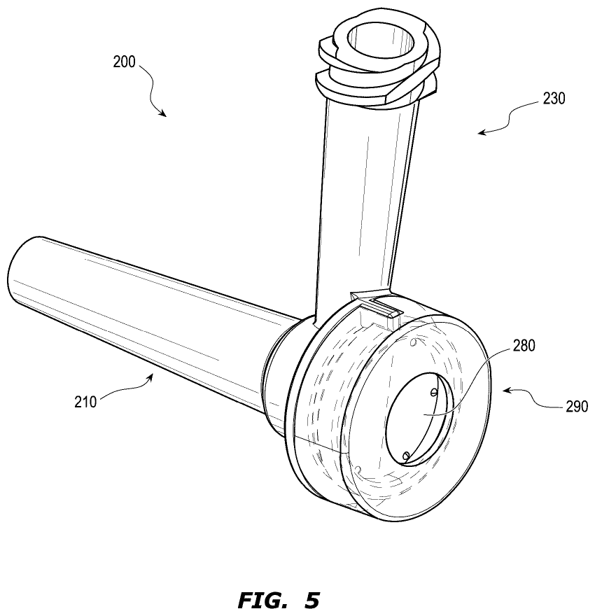

FIGS. 1-4 illustrate an embodiment of a hemostasis valve device that includes a fluid flow insert. FIGS. 5-9 illustrate another embodiment of a hemostasis valve device that includes an offset side-arm. In certain views each device may be coupled to, or shown with, additional components not included in every view. Further, in some views only selected components are illustrated, to provide detail into the relationship of the components. Some components may be shown in multiple views but not discussed in connection with every view. Disclosure provided in connection with any figure is relevant and applicable to disclosure provided in connection with any other figure or embodiment.

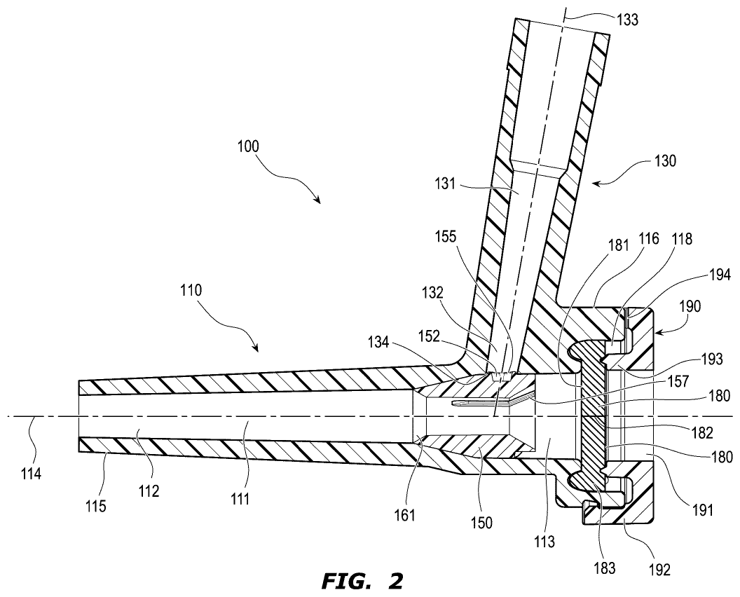

FIGS. 1-4 illustrate an embodiment of a hemostasis valve device 100. As illustrated, the depicted embodiment includes a body member 110, a side-arm 130, a fluid flow insert 150 (shown in FIG. 2), a valve member 180, and a valve cap 190. The valve cap 190 may be fixedly coupled to the body member 110 with the valve member 180 sealingly disposed between the body member 110 and the valve cap 190. The fluid flow insert 150 is co-axially disposed within the body member 110 distally of the valve member 180. The side-arm 130 extends radially outward from the body member 110. The body member 110, side-arm 130, and fluid flow insert 150 can be formed of any suitable translucent or transparent material, such as polycarbonate, acrylic, polypropylene, nylon, copolyester, etc., such that entrapped air bubbles can be visualized within the body member 110, the side-arm 130, and/or a distal surface 181 of the valve member 180. In some embodiments, a length of the hemostasis valve device 100 is between 4 centimeters and 8 centimeters, including between 4 centimeters and 6 centimeters.

As illustrated in FIG. 2, the depicted embodiment includes a body bore 111. The body bore 111 extends from a proximal end to a distal end of the body member 110. The body bore 111 includes a distal bore portion 112 and a proximal bore portion 113. The proximal bore portion 113 is disposed at a proximal end of the body member 110 and proximal to the side-arm 130. A proximal valve portion 118 may be configured to receive the valve member 180 to seal the proximal bore portion 113 to prevent blood and/or fluid from leaking from the body bore 111. The side-arm 130 extends radially outward from the body member 110. A longitudinal axis 133 of the side-arm 130 may be set at an angle ranging from about 45 degrees to about 90 degrees and may be about 80 degrees relative to a longitudinal axis 114 of the body member 110. The side-arm 130 includes a side-arm bore 131 extending therethrough. The side-arm bore 131 includes a distal portion 132 and a side-arm bore outlet 134 that are in fluid communication with the body bore 111. The side-arm 130 may include a fluid fitting (e.g., a female Luer slip or female Luer lock fitting) disposed at a proximal end configured to be coupled to a mating fitting (e.g., male Luer slip or male Luer lock fitting) of a fluid deliver device (e.g., syringe).

FIGS. 1 and 2 illustrate the valve cap 190 coupled to a proximal end of the body member 110. As illustrated in the depicted embodiment, the valve cap 190 includes a circumferential skirt 192 and a proximal opening 191. The skirt 192 may couple with the body member 110 to retain and seal the valve member 180 between the body member 110 and the valve cap 190. A compression ring 193 may apply a compressive force to the valve member 180 to enhance sealing of the valve member 180 with the body member 110. In some embodiments, the body member 110 may include an anti-rotation tab 116 configured to couple with an anti-rotation slot 194 of the valve cap 190. When the anti-rotation tab 116 and the anti-rotation slot 194 are coupled together, the valve cap 190 may be prevented from rotation relative to the body member 110. The valve cap 190 can be formed from any suitable rigid material such as polycarbonate, acrylic, acrylonitrile butadiene styrene, polypropylene, high density polyethylene, polyoxymethylene, nylon, copolyester, etc. In some embodiments, the valve cap 190 may comprise a transparent or translucent material.

FIG. 2 illustrates the valve member 180 disposed between the body member 110 and the valve cap 190. As depicted in the illustrated embodiment, the valve member 180 includes a disk shape having a thickened circumferential ring 183 to facilitate sealing and retention of the valve member 180 between the body member 110 and the valve cap 190. A slit 182 is disposed through a thickness of the valve member 180 in a central portion of the valve member 180. The slit 182 may allow passage of an elongate medical appliance (e.g., guidewire) while sealing around the medical appliance and retaining the medical appliance in a desired position relative to the hemostasis valve device 100. The valve member 180 may be formed of any suitable elastomeric material, such as silicone rubber, rubber, neoprene, thermoplastic elastomer, polyisoprene, polyurethane, etc.

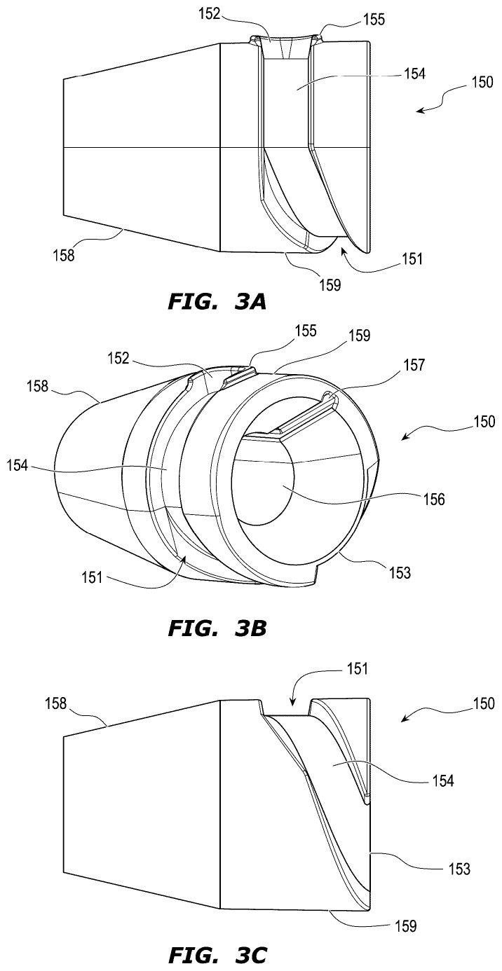

FIGS. 3A-3C illustrate an embodiment of the fluid flow insert 150, in the illustrated embodiment, the fluid flow insert 150 has a generally conical shape with a tapered distal portion 158 and a proximal portion 159. An insert bore 156 extends through the distal portion 158 and the proximal portion 159. The insert bore 156 can be inwardly tapered at the proximal portion 159 to facilitate passage of the elongate medical appliance through the fluid flow insert 150 from the proximal portion 159 to the distal portion 158 (i.e., antegrade passage). The insert bore 156 may include an interior chamfer 161 (shown in FIG. 2) at a distal end to facilitate passage of the elongate medical appliance from the distal portion 158 to the proximal portion 159 (i.e., retrograde passage).

As depicted in the embodiment of the fluid flow insert 150 of FIGS. 3A-3C, the fluid flow insert 150 includes a fluid flow channel 151 disposed in a wall of the proximal portion 159 and in fluid communication with the side-arm bore 131 and the proximal bore portion 113. The fluid flow channel 151 can be configured to increase a speed and/or impose a circular, spiral, or swirling shape fluid flow path to the flushing fluid injected into the proximal bore portion 113 to flush, dislodge, or remove entrapped air bubbles from the proximal bore portion 113 and/or on the distal surface 181 of the valve member 180 when the hemostasis valve device 100 is primed for use. The circular, spiral, or swirling fluid flow path can increase fluid flow speed at or adjacent the surfaces of the proximal bore portion 113, including the distal surface 181. The increased fluid flow speed, the direction of the fluid flow, and/or characteristics of the flow (e.g. the degree of turbulence in the fluid flow) may dislodge air bubbles disposed on the surfaces by creating a higher sheer force at the surface interface. Dislodging air bubbles during priming prepares the system for use and decreases the risk of introduction of air bubbles into the vasculature of the patient.

The fluid flow channel 151 includes an inlet portion 152, an outlet portion 153, and a flow channel 154 extending therebetween and in fluid communication with the inlet portion 152 and the outlet portion 153. The inlet portion 152 is in fluid communication with the side-arm bore 131. A protrusion 155 partially surrounds the inlet portion 152. The protrusion 155 may be at least partially disposed within the side-arm bore outlet 134 to orient the inlet portion 152 to the side-arm bore outlet 134 (shown in FIG. 2). The fluid flow insert 150 may include a longitudinal internal groove 157 configured to couple with a longitudinal boss of an assembly pin to orient the fluid flow insert 150 relative to the body member 110 and the side-arm 130 such that the protrusion 155 is at least partially disposed within the distal portion 132 of the side-arm bore 131 and the inlet portion 152 is in fluid communication with the side-arm bore 131. The outlet portion 153 opens at a proximal end of the proximal portion 159 and is fluid communication with the proximal bore portion 113. The flow channel 154 has a spiral shape that pitches proximally. In other embodiments, the fluid flow channel 151 may have any other suitable shape to direct the flushing fluid in a circular, spiral, swirling flow path within the proximal bore portion 113. For example, the fluid flow channel 151 may have a square or trapezoidal shape. Other shapes are within the scope of this disclosure. A rotation of the flow channel 154 about the circumference of the proximal portion 159 from the inlet portion 152 to the outlet portion 153 may extend between about 90 degrees to about 300 degrees, and may include about 180 degrees to about 270 degrees. The flow channel 154 can have a depth into a wall of the proximal portion 159 ranging from about 0.25 millimeter to about 1.02 millimeters, including from about 0.38 millimeter to about 0.64 millimeter. A cross-sectional area of the flow channel 154 may be smaller than a cross-sectional area of the side-arm bore 131 to facilitate a higher fluid flow speed at the outlet portion 153 relative to the inlet portion 152.

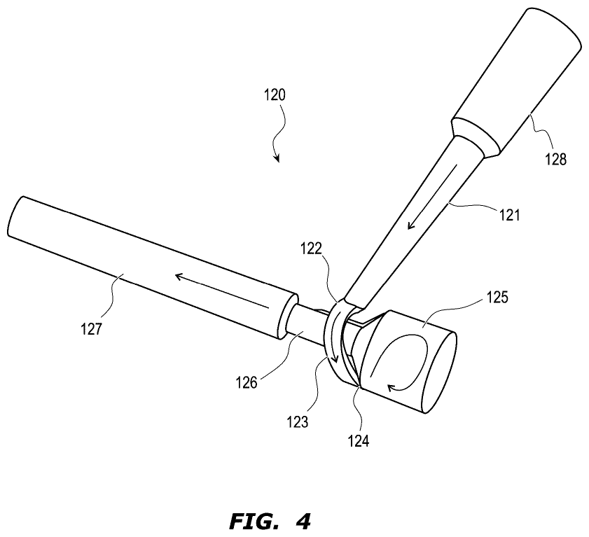

In some embodiments, a distal end 115 of the hemostasis valve device 100 can be coupled to a catheter that has been inserted into a patient's vascular system to provide access to treat the patient's brain or other treatment site. During use, the hemostasis valve device 100 can be primed with the priming or flushing fluid prior to insertion of the catheter into the patient's vascular system. In certain instances, the hemostasis valve device 100 and the catheter are primed together. The priming may be accomplished by coupling a fluid delivery device (e.g., syringe) filled with the flushing fluid (e.g., saline) to the side-arm 130 such the fluid delivery device is in fluid communication with the side-arm bore 131. The flushing fluid can be injected from the fluid delivery device into the hemostasis valve device 100 to prime or fill the body bore 111 and the side-arm bore 131 with the flushing fluid. The injected flushing fluid can form a fluid flow path 120 within the hemostasis valve device 100 as shown in FIG. 4. The fluid flow path 120 includes a fluid delivery device segment 128, a side-arm bore segment 121, an inlet segment 122, a flow channel segment 123, an outlet segment 124, a proximal bore portion segment 125, an insert bore segment 126, and a distal bore segment 127. The fluid speed within the flow channel segment 123 may be faster than the fluid speed within the inlet segment 122. The proximal bore portion segment 125 may include a spiral, circular, or swirling shape fluid flow path configured to dislodge air bubbles from the proximal bore portion 113 and/or distal surface 181.

FIGS. 5-9 depict an embodiment of a hemostasis valve device 200 that resembles the hemostasis valve device 100 described above in certain respects. Accordingly, like features are designated with like reference numerals, with the leading digit incremented to “2.” For example, the embodiment depicted in FIG. 5 includes a body member 210 that may, in some respects, resemble the body member 110 of FIG. 1. Relevant disclosure set forth above regarding similarly identified features thus may not be repeated hereafter. Moreover, specific features of the body member 110 and related components shown in FIGS. 1-4 may not be shown or identified by a reference numeral in the drawing or specifically discussed in the written description that follows. However, such features may clearly be the same, or substantially the same, as features depicted in other embodiments and/or described with respect to such embodiments. Accordingly, the relevant descriptions of such features apply equally to the features of the hemostasis valve device 200 and related components depicted in FIGS. 5-9. Any suitable combination of the features, and variations of the same, described with respect to the hemostasis valve device 100 and related components illustrated in FIGS. 1-4 can be employed with the hemostasis valve device 200 and related components of FIGS. 5-9, and vice versa.

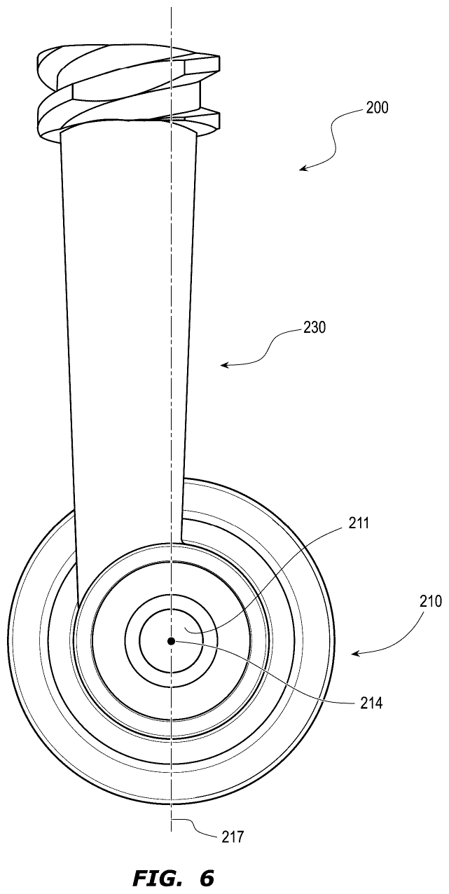

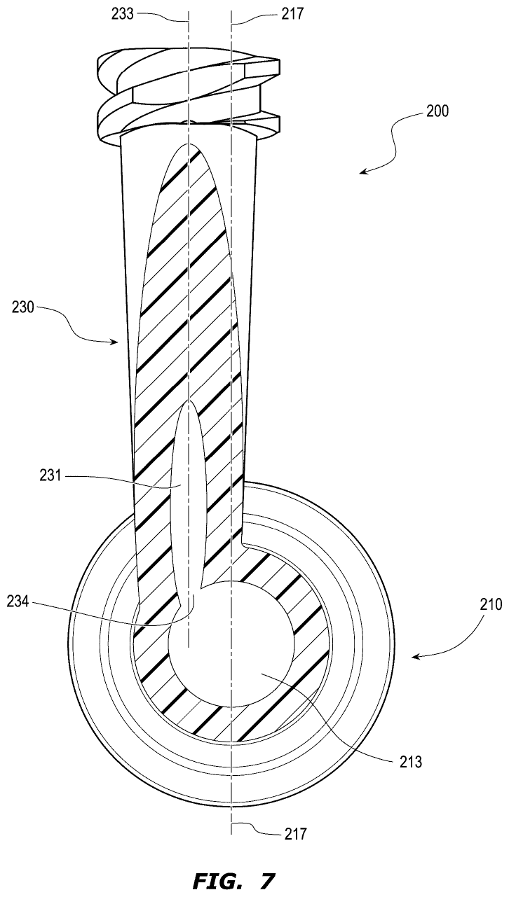

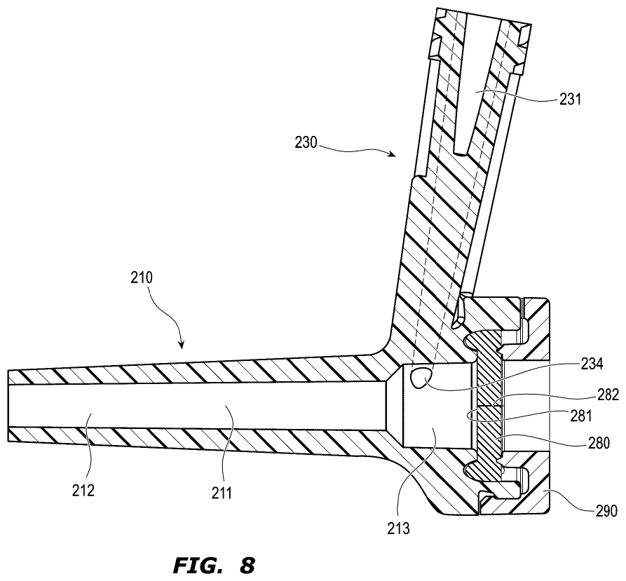

As illustrated in FIG. 5, the depicted embodiment of the hemostasis valve device 200 includes a body member 210, a side-arm 230, a valve member 280, and a valve cap 290. The valve cap 290 is coupled to the body member 210 with the valve member 280 sealingly disposed between the body member 210 and the valve cap 290. The side-arm 230 extends radially outward from the body member 210. As shown in FIG. 6, the side-arm 230 is substantially parallel to and offset from a vertical plane 217 extending through a longitudinal axis 214 of the body member 210 such that a side-arm bore outlet 234 of a side-arm bore 231 is offset from the vertical plane 217, as depicted in FIGS. 7 and 8. In other words, a longitudinal axis 233 of the side-arm bore 231 is substantially parallel to and offset from alignment with the vertical plane 217. This configuration can facilitate flow of priming or flushing fluid from the side-arm bore 231 into the body bore 211 to include a circular, spiral, or swirling shape flow path, and/or otherwise direct fluid flow within a proximal bore portion 213 to flush, dislodge, or remove entrapped air bubbles from the proximal bore portion 213 and/or a distal surface 281 of the valve member 280. The circular, spiral, swirling shape flow path may increase a sheer force of the flushing fluid adjacent a surface of the proximal bore portion 213 and/or distal surface 281 of the valve member 280.

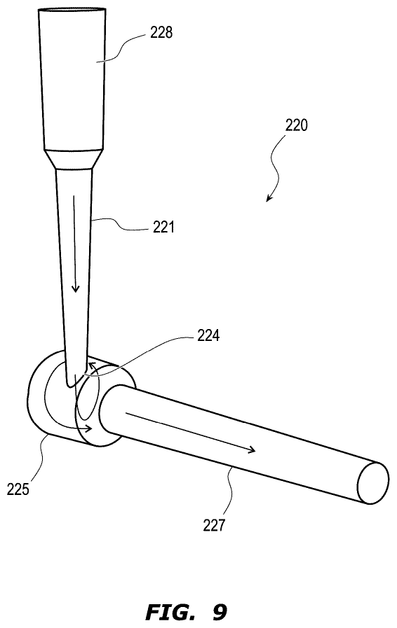

During use, the hemostasis valve device 200 can be primed with the priming or flushing fluid. The priming may be accomplished by coupling a fluid delivery device (e.g., syringe) filled with the flushing fluid (e.g., saline) to the side-arm 230 such that the fluid delivery device is in fluid communication with the side-arm bore 231. The flushing fluid can be injected from the fluid delivery device into the hemostasis valve device 200 to prime or fill the body bore 211 and the side-arm bore 231 with the flushing fluid. The injected flushing fluid can form a fluid flow path 220 within the hemostasis valve device 200 as shown in FIG. 9. The fluid flow path 220 may include a fluid delivery device segment 228, a side-arm bore segment 221, a side-arm bore outlet segment 224, a proximal bore portion segment 225, and a distal bore segment 227. The proximal bore portion segment 225 may include a spiral, circular, or swirling shape fluid flow path configured to dislodge air bubbles from the proximal bore portion 213 and/or distal surface 281 of the valve member 280.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of priming a hemostasis valve may include one or more of the following steps: injecting fluid through a side-arm bore into a proximal body bore portion, wherein the proximal body bore portion includes a circular, spiral, or swirling shape flow path. Other steps are also contemplated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a hemostasis valve device of this disclosure, the proximal end of the device refers to the end nearest to the cap and the distal end refers to the opposite end, the end nearest the fluid fitting.

“Fluid” is used in its broadest sense, to refer to any fluid, including both liquids and gases as well as solutions, compounds, suspensions, etc., that generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially parallel” is recited with respect to a feature, it is understood that in further embodiments, the feature can have a precisely parallel configuration.

The terms “a” and “an” can be described as one, but not limited to one. For example, although the disclosure may recite a body member having “a side-arm,” the disclosure also contemplates that the body member can have two or more side-arms.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A hemostasis valve device, comprising:
a body member comprising a body bore extending therethrough, wherein the body bore comprises a distal bore portion and a proximal bore portion;
a side-arm extending radially outward from the body member and comprising a side-arm bore extending therethrough and in fluid communication with the proximal bore portion; and
a valve member disposed within the body bore proximally of the side-arm and configured to selectively restrict fluid flow through the body bore,
wherein the proximal bore portion comprises a first fluid flow path configured to flush air bubbles from the proximal bore portion.

2. The hemostasis valve device of claim 1, wherein the first fluid flow path is configured to flush air bubbles from a distal surface of the valve member.

3. The hemostasis valve device of claim 1, further comprising a fluid flow insert co-axially disposed within the proximal bore portion distally of the valve member.

4. The hemostasis valve device of claim 3, wherein the fluid flow insert comprises:
a flow channel comprising:
an inlet portion in fluid communication with the side-arm bore;
an outlet portion in fluid communication with the proximal bore portion; and
a flow channel having a spiral shape and extending between and in fluid communication with the inlet portion and the outlet portion.

5. The hemostasis valve device of claim 4, wherein the fluid flow insert further comprises a protrusion at least partially surrounding the inlet portion and configured to be at least partially disposed within a distal portion of the side-arm bore.

6. The hemostasis valve device of claim 4, further comprising a first fluid flow path comprising:
a first side-arm bore segment extending through the side-arm bore;
an inlet segment extending through the inlet portion;
a channel segment extending through the flow channel;
an outlet segment extending through the outlet portion; and
a first proximal bore segment comprising a first circular or spiral or swirl shape path.

7. The hemostasis valve device of claim 4, wherein the fluid flow insert comprises an insert bore extending through the fluid flow insert and in communication with the proximal bore portion and the distal bore portion.

8. The hemostasis valve device of claim 1, wherein the side-arm is offset from a vertical plane aligned with a longitudinal axis of the body member.

9. The hemostasis valve device of claim 8, wherein the side-arm bore comprises a side-arm bore outlet in fluid communication with the proximal bore portion and disposed offset from the vertical plane.

10. The hemostasis valve device of claim 9, further comprising a second fluid flow path comprising:
a second side-arm bore segment extending through the side-arm bore;
a side-arm bore outlet segment extending through the side-arm bore outlet; and
a second proximal bore segment comprising a second circular or spiral or swirl shape path.

11. A hemostasis valve device, comprising:
a body member comprising a body bore extending through the body member, wherein the body bore comprises a distal bore portion and a proximal bore portion;
a side-arm extending radially outward from the body member and comprising a side-arm bore extending through the side-arm and in fluid communication with the body bore;
a valve member disposed within the body bore proximally of the proximal bore portion and configured to selectively restrict fluid flow through the body bore; and
a fluid flow insert co-axially disposed within the body bore distally of the valve member,
wherein the fluid flow insert is configured to direct a fluid flowing within the proximal bore portion in a circular flow path to flush air bubbles from the proximal bore portion.

12. The hemostasis valve device of claim 11, wherein the fluid flow insert is configured to direct the fluid flowing within the proximal bore portion in a circular or spiral or swirling shape flow path to flush air bubbles from a distal surface of the valve member.

13. The hemostasis valve device of claim 11, wherein the fluid flow insert comprises:
a flow channel comprising:
an inlet portion in fluid communication with the side-arm bore;
an outlet portion in fluid communication with the proximal bore portion; and
a flow channel having a spiral shape extending between and in fluid communication with the inlet portion and the outlet portion.

14. The hemostasis valve device of claim 13, wherein the fluid flow insert further comprises a protrusion at least partially surrounding the inlet portion and configured to be at least partially disposed within a distal portion of the side-arm bore.

15. The hemostasis valve device of claim 13, further comprising a first fluid flow path comprising:
a first side-arm bore segment extending through the side-arm bore;
an inlet segment extending through the inlet portion;
a flow channel segment extending through the flow channel;
an outlet segment extending through the flow channel; and
a first proximal bore segment extending through the proximal bore portion comprising a first circular or spiral or swirl shape path.

16. The hemostasis valve device of claim 13, wherein the fluid flow insert comprises an insert bore extending through the fluid flow insert and in communication with the proximal bore portion and the distal bore portion.

17. A method of removing air bubbles from a medical device, comprising:
injecting fluid through a side-arm bore of the medical device into a proximal body bore portion of the medical device,
wherein the proximal body bore portion includes a circular, spiral, or swirling shape flow path.

18. The method of claim 17, wherein injecting fluid the through the side-arm bore of the medical device into the proximal body bore portion of the medical device comprises injecting the fluid through a fluid flow channel of a fluid flow insert, wherein the fluid flow channel comprises a spiral shape.

19. The method of claim 17, wherein injecting fluid through the side-arm bore of the medical device into the proximal body bore portion of the medical device comprises injecting the fluid through a side-arm bore outlet disposed offset from a vertical plane aligned with a longitudinal axis of the proximal bore portion.
